(19) United States
(12) Patent Application Publication
(10) Pub. No.: US 2002/0164571 A1
(43) Pub. Date: Nov. 7, 2002

(54) Method of preserving metanephroi in vitro prior to transplantation

(76) Inventor: Marc R. Hammerman, St. Louis, MO (US)

Correspondence Address: FLEHR HOHBACH TEST ALBRITTON & HERBERT LLP, Four Embarcadero Center, Suite 3400, San Francisco, CA, 94111-4187, US

(21) Appl. No.: 09/798,790

(22) Filed: Mar. 2, 2001

(51) Int. Cl. A61K 45/00

(52) U.S. Cl. 435/2

(57) ABSTRACT

The present invention is directed towards methods of preserving embyronic metanephroi prior to transplantation.

### GOVERNMENT SUPPORT

This invention was made with Government support from the National Institute of Health Grant/Contract Nos. DK45181 and DK53487. The U.S. Government may have certain rights to this invention.

### FIELD OF THE INVENTION

The field of invention is generally related to methods of preserving embryonic metanephric kidney tissue prior to transplantation.

### BACKGROUND OF THE INVENTION

End-stage chronic renal failure afflicts more than 300,000 individuals in the United States alone, most of whom are treated using dialysis, a treatment with considerable morbidity (U.S. Renal Data System, (1999) Am. J. Kidney Dis., 34:S40-S50), or renal allotransplantation, which is limited by the number of organs available to transplant (U.S. Renal Data System, (1999) Am. J. Kidney Dis., 34:S74-S86).

Another possible solution for the lack of organ availability is the transplantation of developing kidneys (metanephric allografts or xenografts). There are at least two reasons why transplantation of allograft (or xenograft) metanephroi into adult animals might be advantageous relative to the transplantation of kidneys. First, for several days following its formation, the metanephros has no vasculature (Saxon L and Sariola H, (1987) Pediatr. Nephrol., 1:385-392), and therefore contains few or no antigen presenting cells derived from the circulation. Depletion of antigen presenting cell would be expected to render allograft metanephroi less immunogenic (Lacy P E, et al., (1981) Diabetes, 30:285-291). Second, the transplanted metanephros becomes a chimeric organ in that it is vascularized in part by blood vessels originating from the host. Rejection that is initiated by antibodies directed against antigens on endothelial cell surfaces, is circumvented to the extent that the transplanted organ is supplied by host vessels.

The possibility that renal function can be enhanced through the addition of functioning nephrons via transplantaion of allograft metanephroi intrarrenally or intraabdominally has been explored (Abrahamson D R, et al., (1991) Lab. Invest., 64:629-639; Robert B, et al., (1996) Am. J. Physiol., 271 :F744-F753; Woolf A S, et al., (1990) Kidney Int., 38:991-997; Barakat T L and Harrison R G (1971) J. Anat., 110: 393-407; Koseki C, et al., (1991) Am. J. Physiol., 261 :C550-C556). However, the results of these investigations indicate that transplantation of metanephroi into adult hosts is complicated by graft rejection (Abrahamson D R, et al., (1991) Lab. Invest., 64:629-639).

Growth factors also have been used for the purpose of reducing transplant rejection and improving transplant function. U.S. Pat. No. 5,135,915 to Czarniecki et al., describes immersing grafts in a formulation comprising transforming growth factor for a period of a few minutes up to several days prior to transplantation. The pretreatment with TGF-β purportedly reduces transplant rejection. U.S. Pat. No. 5,728,676, to Halloran describes the administration of insulin-like growth factor (IGF) before, during, or after organ transplantation for the purpose of inhibiting transplant rejection. In a canine renal autotransplantation model, it was found that storing the removed kidneys in a preservation solution supplemented with IGF-I for a period of 24 hours prior to transplantation back into the dog, significantly improved renal function for the first 5 days following transplantation (Petrinec et al. (1996), Surgery 120(2):221-226).

Another approach to circumventing graft rejection involves implanting embryonic metanephroi subcapsularly into kidneys or into the omentum (Rogers S A, Lowell J A, Hammerman N A, and Hammerman M R, (1998) Kidney International 54:27-37). The survival, growth, maturation, vascularization and function of the metanephroi following transplantation indicate that renal organogenesis occurs and that a vascularized functional chimeric kidney results. The growth and function of transplanted metanephroi was enhanced when metanephroi were exposed to growth factors (Rogers S A, Powell-Braxton L. and Hammerman M R, (1999) Developmental Genetics 24:293-298, Hammerman M R, (2000) Kidney International 57: 742-755).

To the extent that transplanted metanephroi are more vascularized by the host and elicit an muted immune response relative to transplanted, developed kidneys, metanephroi may be an attractive alternative to the use of human renal allografts. Accordingly, it would be useful to develop methods for the in vitro preservation of metanephroi, similar to that used for renal allografts, for a period of time prior to transplantation.

### SUMMARY OF THE INVENTION

In accordance with the objects outlined above, the present invention provides a method of preserving embryonic metanephric tissue in embryonic metanephric tissue (EMT) preservation solution. Tissue so preserved develops into a functional chimeric kidney upon transplantation.

The invention also provides a method for preserving embryonic metanephric tissue in an EMT preservation solution modified by the addition or substitution of agents which minimize injury due to storage in the preservation solution and/or enhance the function of the tissue following transplantation. In particular, growth factors may be added to EMT preservation solution.

Methods are also disclosed for optimal temperatures and times for preserving metanephroi.

### BRIEF DESCRIPTION OF THE DRAWINGS

FIG. 1 depicts photomicrographs of hematoxylin & eosin-stained mid-sagittal sections of metanephroi originating from an E15 rat embryo (a,b); originating from an E15 rat embryo following 3 days of preservation in UW solution (c,d); originating from an E16 rat embryo (e,f); or originating from an E13 metanephros grown in organ culture for 3 days (g, h). Shown are metanephric blastema (MB), branched segments of ureteric bud (UB) and developing nephron S-shaped bodies (S). Arrows delineate the nephrogenic zone. Photomicrographs are representative of >10 experiments. Magnifications are shown for a, c, e and g (a), and for b, d, f, and h (b).

FIG. 2 depicts photomicrographs of hematoxylin & eosin-stained sections of developed E15 metanephros 4 weeks following implantation in a host peritoneum. The metanephros was preserved in UW solution for 3 days prior to implantation. a) Shown is a ureter (u); b) Shown is a glomerulus (g), a proximal tubule (pt) with its brush border membrane delineated (arrowhead) and a distal tubule (dt); c) Shown are mature collecting ducts (cd). Photomicrographs are representative of >10 experiments. Magnifications are shown for (a) and (b) and (c).

### DETAILED DESCRIPTION OF THE INVENTION

The present invention allows for the preservation of embryonic metanephroi prior to transplantation into a recipient. Following surgical removal, metanephroi are placed in an preservation solution. The preservation solution is designed to minimize injury during preservation such that functional chimeric kidneys develop from metanephroi following transplantation into a recipient.

Although, no direct analogy is possible between the preservation of metanephroi and kidneys, the composition of the preservation solution used to preserve metanephroi is based on principles known to minimize preservation injury to human renal allografts (McKay D B, Milford E L, and Sayegh M H. Clinical aspects of renal transplantation. In: The Kidney: Physiology and Pathophysiology. Edited by B. M. Brenner Philadelphia. W. B. Saunders, 1996, p. 2602-2652).

The methods for isolating and transplanting embryonic methanephroi described in U.S. Pat. No. 5,976,524 and U.S. Ser. No. 09/222,460 are applicable here and are incorporated by reference in their entirety.

Accordingly, the present invention is drawn to methods of preserving metanephroi in an “embryonic methanephroi tissue” (EMT) preservation solution. The EMT preservation solution used to preserve metanephroi should minimize preservation injury. Preferably, the composition of the EMT preservation solution includes factors which contribute to the development of functional chimeric kidneys from metanephroi following transplantation.

Injury due to preservation may be minimized by designing EMT preservation solutions. The initial choice of constituents may be modeled after those found in existing preservation solutions. For example, University of Wisconsin (UW) preservation solution has a number of constituents. These constituents have been proposed to have properties beneficial for the preservation of organs (see Brennan, D C and Lowell, J A. Pre-transplant preparation of the cadaver donor/organ procurement. In: Primer on Transplantation. Edited by D J Norman and W N Suki, Am. Soc. Transplant Physicians Press, Thorofare, 1998, pp. 197-204). Constituents found in UW solution proposed to minimize hypothermia induced osmotic swelling and sodium pump paralysis include lactobionate, raffinose and hydroxyethyl starch. Constituents proposed to minimize expansion of the interstitial space include hydroxyethyl starch. Constituents which are included because they are thought to decrease the generation of oxygen-free radicals are allopurinol and/or glutathione. To decrease or prevent the depletion of high-energy phosphate compounds, UW solution contains adenosine as an constituent.

Alternatively, certain constituents are preferably avoided. For example, glucose is preferably not included in UW solution in order to minimize intracellular acidosis.

In a preferred embodiment, the EMT preservation solution is UW solution, pH 7.4 (marketed under the name ViaSpan®, DuPont Parmaceuticals) and has the composition shown in Table 1.

TABLE 1
Composition of EMT Preservation Solution

| | |
|---|---|
| Compound | Amount per Liter |

| | | |
|---|---|---|
| Potassium lactobionate | 100 | mM |
| KH₂PO₄ | 25 | mM |
| MgSO₄ | 5 | mM |
| Raffinose | 30 | mM |
| Adenosine | 5 | mM |
| Gluthathione | 3 | mM |
| Insulin | 40 | U/L* |
| Penicillin | 200,000 | U/L* |
| Dexamethasone | 8 | mg/L* |
| Allopurinol | 1 | mM |
| Hydroxyethyl starch | 50 | g/L |

*Compounds added just prior to use

The EMT preservation solution may be modified by omitting, substituting or adding compounds to improve its “preservative quality”. “Preservative quality” herein refers to components which enhance the development and differentiation of metanephroi following transplantation. Generally, compounds are chosen based on properties which extend the length of time that embryonic metanephroi may be stored in the EMT preservation solution prior to transplantation.

For example, in some embodiments, agents, such a polyethylene glycol, may be substituted for hydroxyethyl starch in the EMT preservation solution of Table 1. In other embodiments, compounds such as electrolytes, triiodothyronine, and cortisol may be added to improve the preservative qualities of the EMT solution.

Such modifications to the EMT solution can be tested by comparing renal function and/or development of metanephroi preserved in EMT solution having the composition shown in Table 1, e.g., UW solution, with metanephroi preserved in a EMT solution which has been modified through the addition or deletion of a component. Preferably the modification improves the preservative quality of the EMT solution.

EMT preservation solutions also are designed to enhance the function of embryonic metanephroi upon transplantation into a recipient. In other words, compounds may be added to the EMT preservation solution which promote renal development and/or function following transplantation into a recipient. Renal development may be judged by kidney weight, vascularization and formation of kidney tissue, e.g., glomeruli, tubules, renal papilla and ureter. Kidney function can be determined by inulin or creatinine clearance.

In a preferred embodiment, the EMT preservation solution is modified by the addition of growth factors. As used herein, the phrase “growth factor” for use in the preservation of metanephroi refers to any molecule that promotes renal development or function of metanephric tissue upon transplantation. Thus, the phrase encompasses growth factors which promote the growth, proliferation, and/or differentiation of metanephric tissue.

Preferred growth factors include tamm horsfall glycoprotein (THG), ligands of the EGF-receptor such as transforming growth factor alpha (TGFα), insulin-like growth factors (IGFs), particularly IGF-I and IGF-II; fibroblast growth factors, particularly basic fibroblast growth factor (bFGF), vitamin A and derivatives thereof such as retinoic acid; vascular endothelial growth factor (VEGF); hepatocyte growth factor (HGF), nerve growth factor (NGF), transferrin, prostaglandin E₁ (PGE₁), human recombinant interleukin 10, and corticotropin releasing hormone (CRH).

It is intended that each of the terms used to define metanephric growth factors includes all members of a given family. For example, the fibroblast growth factor family consists of at least 15 structurally related polypeptide growth factors (Szebenyi and Fallon (1999) Int. Rev. Cytol., 185:45-106).

Other growth factors which may be included are epidermal growth factor (EGF), and amphiregulin; growth hormone, platelet-derived growth factor, leukemia inhibitory factor (LIF), angiopoetins 1 and 2, and bone morphogenetic proteins (BMPs), cytokines such as TGF-β and other members of the TGF-β family (see Atrisano et al. (1994), J. Biochemica et Biophysica Acta 1222:71-80), growth hormone (GH) (see Hammerman, M. R. (1995), Seminars in Nephrology) and sodium selenite.

In a preferred embodiment, one of more of the following growth factors is added to EMT preservation solution: IGF I; IGF II; TGFα; HGF; VEGF; bFGF; NGF; RA; CRH; THG; prostaglandin E1, and iron saturated transferrin. Concentrations between about 1 ng/ml to 10 μg/ml are usually sufficient for most growth factors. The concentration of a given growth factor can be optimized using titration experiments.

Using known procedures, it can readily be determined whether the addition, substitution or omission of a compound increases the time which embryonic metanephroi can be preserved and/or promotes renal function of preserved metanephroi upon transplantation into a recipient. For example, comparisons of size and extent of tissue differentiation can be made between metanephroi implanted directly to metanephroi stored in EMT preservation solution. Differences, if any, in renal function between preserved metanephroi and metanephroi implanted directly can be detected by measuring inulin clearances and urine volumes.

In addition, TUNEL stains on fixed metanephroi post-preservation may by used to determine the effect which a given growth factor(s) has on enhancing development and differentiation of metanephroi (Sorenson, et al. (1995) Am. J. Physiol., 268:F73-F81).

The methods of the invention are used to preserve embryonic metanephric kidney so that functional chimeric kidneys develop following transplantation into a recipient. By “preserved” herein is meant storing isolated embyonic metanephroi for a period of time prior to transplantation. Conditions critical to preservation include the duration of warm and cold ischemia.

Generally, the duration of “warm ischemia for metanephroi” defined herein as the time required to surgically remove metanephroi, will be below 20 minutes. In a preferred embodiment, the duration of warm ischemia is minimized by keeping the duration of warm ischemia to 15 minutes or less.

Similarly, the duration of“cold ischemia for metanephroi”, defined herein as the length of time which the metanephroi may be stored in an EMT preservation solution is less than 14 days. In a preferred embodiment, the duration of cold ischemia is three days.

It is important that the temperature of the EMT preservation solution used to preserve embryonic metanephroi be cold. In a preferred embodiment, the temperature of the EMT preservation solution is maintained between 0 to 4° C. In an especially preferred embodiment, EMT preservation solution is ice cold. Ice-cold EMT solutions are obtained by placing the EMT preservation solution in an ice bath.

Prior to preservation, metanephric tissue is harvested from one or more suitable mammalian donors at an appropriate stage of fetal development. Preferably, the metanephric tissue is harvested soon after the metanephric kidney begins formation and prior to the presence of blood vessels that either originate within the metanephros or from inside or outside the metanephros. Tissue harvested too late in the development of the metanephric kidney, for example, tissue having visible blood vessels, may contain more antigen-presenting cells and cell-surface antigens and thus present more of threat of rejection by the recipient. Preferably, the harvested metanephroi contains metanephric blastema, segments of ureteric bud, and nephron precursors, and does not contain glomeruli.

The preferred developmental stage for harvesting the metanephros will vary depending upon the species of donor. Generally, the metanephros is preferably harvested 1 to 5 days after the metanephros forms. Preferably, the metanephros is harvested from 1 to 4 days after the metanephros forms, and more preferably from about 2 to 4 days after metanephros formation. In rats, the metanephros forms on day 12.5 of a 22-day gestation period, and on day 11 of a 19 day gestation period in mice. In these species, a suitable time frame in which to harvest the donor metanephros of mice or rats is typically between the second and fourth day after the metanephros begins formation. Preferably the metanephros is harvested within 3 days after formation of the metanephros begins.

In species having a longer gestation period, the time-frame during which the metanephros is preferably harvested following its formation, can be longer. Generally, the time frame in which the metanephros is harvested will be less than about one fifth of the total gestation period of the donor, preferably less than about one seventh of the total gestation period of the donor, and more preferably, less than about one tenth of the total gestation period of the donor. Table 2 shows the time-course (in days) of metanephros development and gestational period in some vertebrates.

TABLE 2

| | Metanephros | Gestational |
|---|---|---|
| | Formation (days) | Period (days) |
| Human | 35-37 | 267 |
| Macaque | 38-39 | 167 |
| Pig | 20-30 | 114 |
| Guinea Pig | 23 | 67 |
| Rabbit | 14 | 32 |
| Rat | 12.5 | 22 |
| Mouse | 11 | 19 |
| Hamster | 10 | 16 |

Pigs are preferred xenogeneic donors for humans because of their comparable organ size, and availability. Additionally, the digestive, circulatory, respiratory and renal physiologies of pigs are very similar to those of humans. In the case of renal function, the maximal renal concentrating ability (1080 mOsm 1⁻¹), total renal blood flow (3.0-4.4 ml min⁻¹ g⁻¹) and glomerular filtration rates (126-175 ml min⁻¹ 70 kg) of the miniature pig are virtually identical to those of humans (see Sachs D H (1994), Veterinary Immunology and Immunopathology 43: 185-191). The use of metanephroi from transgenic pigs that have been “humanized” to reduce the potential for transplant rejection may provide further advantages (e.g. Pierson et al. (1997), J. Heart Lung Transplant 16:231-239). Pig metanephroi are harvested at about the 10 mm stage. This occurs between approximately embryonic day 20 and embryonic day 30. Human tissue could be used as an allogeneic source for transplantation.

Metanephroi are removed surgically as described previously(see above and Rogers et al. (1998), Kidney Int., 54:27-37), and placed in EMT preservation solution until they are transplanted. It is preferred to use the whole metanephros, with renal capsule intact, for transplantation. One or more metanephroi may be used per recipient, depending upon the increase in nephron mass that the recipient needs.

To transplant the metanephric tissue, surgery is performed on the recipient to expose one or both kidneys. Surgical procedures for the transplantation of metanephroi are well known in the art (e.g. Rogers S A, Lowell J A, Hammerman N A, and Hammerman M R, (1998) Kidney International 54:27-37; Rogers S A, Powell-Braxton L. and Hammerman M R, (1999) Developmental Genetics 24:293-298, Hammerman M R, (2000) Kidney International 57: 742-755).

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference.

### EXAMPLES

### Example 1

### Preservation of Embryonic Metanephroi

### Methods

Metanephroi were surgically dissected from E15 Sprague-Dawley rat embryos under a dissecting microscope using previously described techniques (Rogers S A, Lowell J A, Hammerman N A, and Hammerman M R., (1998) Kidney International 54:27-37) and placed in ice-cold in UW solution. When indicated, the following growth factors, previously shown to enhance the function of transplanted metanephroi (Hammerman M R., (2000) Kidney International 57: 742-755; Hammerman M R., (2000) Pediatric Nephrology 14:513-517; Rogers S A, Powell-Braxton L., and Hammerman M R., (1999) Developmental Genetics 24:293-298) were added to the UW solution (UW+ growth factors): recombinant human insulin-like growth factor I (IGF I) (Genentech Inc. S. San Francisco Calif.), 10⁻⁷ M; recombinant human IGF II (Bachem Inc., Torrance Calif.), 10⁻⁷ M; recombinant human transforming growth factor α (TGFα) (Upstate Biotechnology Inc. Lake Placid N.Y.), 10⁻⁸ M; recombinant human hepatocyte growth factor (HGF) (R&D Systems, Minneapolis Minn.), 10₋₈ M; recombinant human vascular endothelial growth factor (VEGF) (Genentech Inc.), 5 ug/ml; recombinant human basic fibroblast growth factor (bFGF) (R&D Systems), 5 ug/ml; recombinant human nerve growth factor (NGF) (Boehringer Mannheim, Indianapolis Ind.), 5 ug/ml; retinoic acid (RA) (Sigma Chemicals, St. Louis Mo.), 10⁻⁶ M; corticotropin releasing hormone (CRH), (Sigma Chemicals) 1 ug/ml; Tamm Horsfall protein (THP) (Biomedical Technologies Inc. Stoughton, Mass.), 1 ug/ml; 25 mM prostaglandin E1, and iron-saturated transferrin (5 ug/ml).

Some metanephroi were implanted directly in the omentum of anaesthetized 6 week old female (host) Sprague Dawley rats after 45 minutes of incubation on ice in UW solution or UW solution+ growth factors. Others were implanted after 3 days in of storage in a 2 ml sterile screw cap sterile plastic microcentrifuge tube (Fisher, Houston Tex.) containing 1 ml of ice-cold UW solution or UW+ growth factors. During the same surgery, host rats had one kidney removed.

When indicated, four weeks following transplantation, end-to-end ureteroureterostomy was performed using microvascular technique (interrupted 10-0 suture) between the ureter of a metanephros implanted in the omentum and the ureter of the kidney that had been removed. Eight weeks later all remaining native renal tissue (the contralateral kidney) was removed from host rats, following which inulin clearances were measured on conscious rats after placement of an indwelling bladder catheter and intravenous line exactly as in previous studies (Rogers S A, Lowell J A, Hammerman N A, and Hammerman M R., (1998) Kidney International 54:27-37).

Baseline measurements for inulin were performed on urine and blood samples obtained prior to beginning the inulin infusions. These “background” values were subtracted from measurements performed after beginning the inulin infusion. Infusion was begun only following removal of all remaining native renal tissue and drainage of all urine remaining in the bladder (10-20 μl). Only the implanted metanephros remained connected to the bladder. As before, rats received no immunosuppression (Rogers S A, Lowell J A, Hammerman N A, and Hammerman M R., (1998) Kidney International 54:27-37).

Metanephroi were fixed, embedded in paraffin, sectioned, and stained with hematoxylin and eosin exactly as in previous studies (Rogers S A, Lowell J A, Hammerman N A, and Hammerman M R., (1998) Kidney International 54:27-37).

Organ culture of E13 metanephroi was carried out in a Dulbecco's modified Eagles Medium: Hams F12 (DMEM:HF12) solution exactly as previously described (Rogers S A, Ryan G, and Hammerman M R., (1991) J. Cell. Biol., 113:1447-1454, Rogers S A, Ryan G, and Hammerman M R., (1992) Am. J. Physiol. Renal Fluid Electrolyte Physiology 262:F533-F539).

Multiple comparisons in of data shown in Table 3 were made using Student-Newman-Keuls Multiple Comparison Test.

### Results

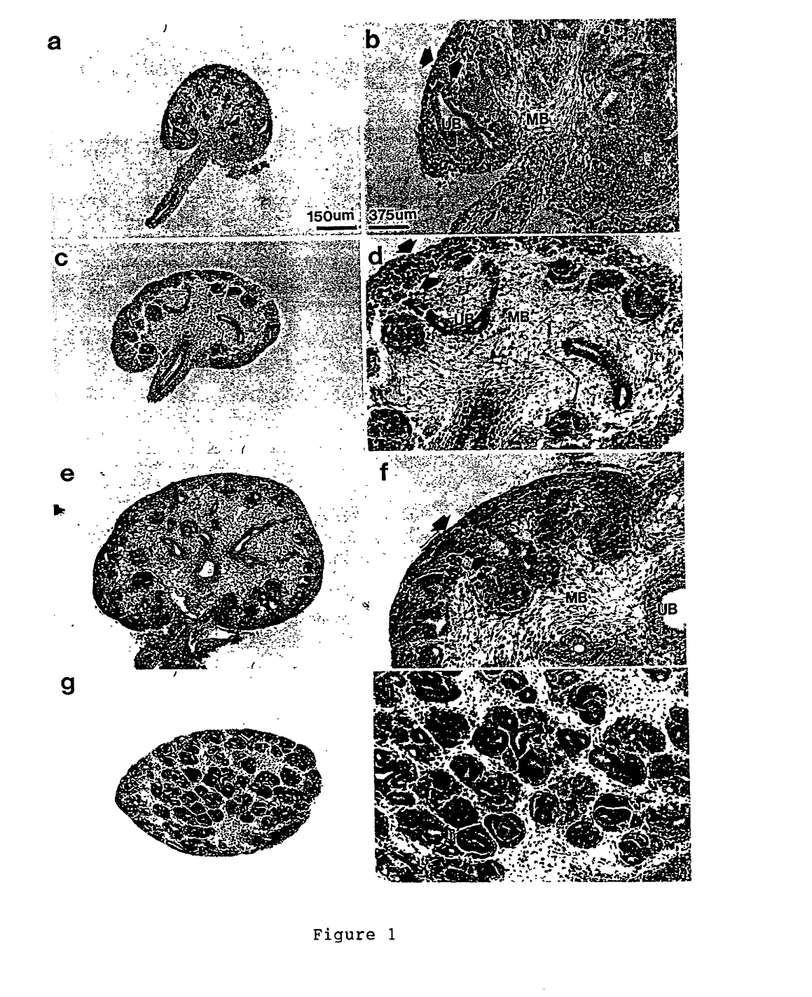

Shown in FIG. 1a, c, e and g are photomicrographs of hematoxylin & eosin-stained sections of rat metanephroi. Higher power view are provided in FIG. 1b, d, f, and h for metanephroi shown in FIG. 1a, c, e, and g respectively. FIG. 1a and b illustrate an E15 metanephros consisting largely of undifferentiated metanephric blastema (MB), and branches of ureteric bud (UB). A nephrogenic zone is delineated by arrows. FIG. 1c and d illustrate an E15 metanephros following 3 days of preservation in ice-cold UW solution. The size (mid-sagittal diameter) of the El 5 metanephros following 3 days of preservation (FIG. 1c) is approximately the same as that of the non-preserved E15 metanephros (FIG. 1a). As would be expected since it originates from an embryo 1 day older, an E16 metanephros (FIG. 1e) is larger than the E15 metanephros (FIG. 1a). However, the E16 metanephros (FIG. 1e) is also larger than the E15 metanephros that had been preserved for 3 days (FIG. 1c), and has a wider nephrogenic zone (FIG. 1f arrows) than either the E15 metanephros (FIG. 1b) or the E15 preserved metanephros (FIG. 1d). Like the E15 metanephros shown in FIG. 1b, the E15 preserved metanephros shown in FIG. 1d consists largely of undifferentiated metanephric blastema (MB), and branches of ureteric bud. In contrast, more developed nephron structures, such as a S-shaped body (S) can be delineated in the E16 metanephros (FIG. 1f).

Illustrated in FIG. 1g and h are photomicrographs of an E13 rat metanephros following 3 days in organ culture. While its size is approximately the same as those of metanephroi shown in FIG. 1a and 1c, the state of differentiation of nephron structures, such as the S-shaped body shown in FIG. 1h, comparable to that reported in previous studies (Hammerman M R, Rogers, S A and Ryan G, (1992), Am. J. Physiol. Renal Fluid Electrolyte Physiology 262:F523-F532, 1992; Rogers S A, Padanilam B J, Hruska K A, Giachelli C M, and Hammerman M R., (1997), Am. J. Physiol. Renal Fluid Electrolyte Physiology 272:F469-F476), is more advanced than those shown in the E15 metanephros (FIG. 1b) or the E15 metanephros following 3 days of preservation in vitro (FIG. 1d).

E15 metanephroi that were preserved in UW+growth factors were histologically indistinguishable from E15 metanephroi that were preserved in UW solution (not shown).

The data shown in FIG. 1 do not exclude the possibility that some development takes place in E15 metanephroi during 3 days of preservation in UW solution on ice (chronological age 18 days). However, whatever development does occur is much less than that observed in metanephroi of lower chronological age such as E13 metanephroi following 3 days in organ culture (chronological age 16 days) (FIG. 1g and h) or E16 metanephroi (chronological age 16 days) (FIG. 1e and f).

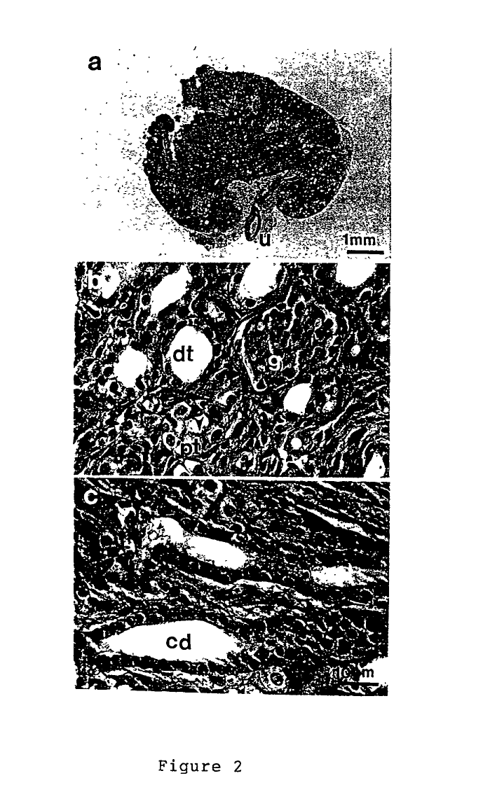

Shown in FIG. 2 are photomicrographs of hematoxylin & eosin-stained sections of a developed E15 metanephros 4 weeks following implantation in the peritoneum of a host rat. The metanephros had been preserved for 3 days in UW solution (no growth factors) prior to implantation.

As we have shown previously for E15 metanephroi that are not preserved in vitro prior to transplantation (Rogers S A, Lowell J A, Hammerman N A, and Hammerman M R., (1998), Kidney International 54:27-37), transplanted developed preserved metanephroi are kidney-shaped (FIG. 2a). A ureter (u) is present. Cortices contain mature-appearing glomeruli (g) and proximal (pt) and distal (dt) tubules (FIG. 2b). The medulla contains mature collecting ducts (cd) (FIG. 2c).

Weights, urine volumes and inulin clearances were measured at 12 weeks post-transplantation, in metanephroi that had been transplanted directly, or transplanted following 3 days of preservation in UW solution with or without growth factors.

The addition of growth factors to the UW solution (compared to no growth factors) increased the weights of metanephroi transplanted directly, but not following 3 days of preservation (Table 3). Urine volumes measured at the time of inulin clearances were significantly increased compared to all other groups by the addition of growth factors to the UW solution of preserved metanephroi.

Inulin clearances were expressed as ul/min/100 g rat weight. Rat weights did not vary between groups at the time inulin clearances were measured (Table 3). Clearances of developed metanephroi transplanted directly (0.43±0.06 ul/min/100 g) or after 3 days of preservation in UW solution without growth factors (0.38±0.08 ul/min/100 g) were comparable to clearances previously measured in Sprague-Dawley to Sprague-Dawley transplants (Rogers S A, Lowell J A, Hammerman N A, and Hammerman M R., (1998) Kidney International 54:27-37; Rogers S A, Powell-Braxton L. and Hammerman M R., (1999) Developmental Genetics 24:293-298; Rogers S A, Liapis H, and Hammerman M R., (2001) Am. J. Physiol. Regulatory Integrative Comparative Physiology 280: R132-R136). Addition of growth factors to the UW solution increased inulin clearances measured in developed metanephroi that had been implanted directly (1.1±0.2 ul/min/100 g) or after 3 days of preservation in vitro (1.2±0.2 ul/min/100 g) compared to clearances measured in either group of metanephroi that were not exposed to growth factors (Table 3).

TABLE 3
Weights, urine volumes and inulin clearances of metanephroi (Met)

| | Transplanted Directly | | | |
|---|---|---|---|---|
| Growth Factors | (TD) | | Preserved for 3 Days (P3) | |
| (GF) | None | GF | None | GF |
| Number | 4 | 5 | 4 | 4 |
| Met Weight | 66 ± 19 | 107 ± 6.0^{a} | 51 ± 7.5 | 75 ± 12 |
| (mg) | | | | |
| Host Weight | 245 ± 8.8 | 268 ± 8.1 | 267 ± 7.6 | 256 ± 5.9 |
| (g) | | | | |
| Urine vol. | 51 ± 8.1 | 96 ± 8.1 | 57 ± 13 | 140 ± 25^{b,c,d} |
| (μl/hr) | | | | |
| Inulin | 0.43 ± 0.06 | 1.1 ± 0.2^{a,e} | 0.38 ± 0.08 | 1.2 ± 0.2^{b,f} |
| Clearance | | | | |
| (μl/min/ | | | | |
| 100 mg) | | | | |

Data are presented as mean± SEM; ^{a}TD GF>P3, p<0.05; ^{b}P3 GF>TD, p<0.05; ^{c}P3 GF>P3, p<0.05; ^{d}P3 GF>TD GF, p<0.05; ^{e}TD GF>TD, p<0.05; ^{f}P3 GF>P3, p<0.05.

### Example 2

### Effect of Growth Factors on the Preservation of Embryonic Metanephroi

### Methods

Metanephroi were surgically dissected as described in Example 1 and placed in ice-cold UW solution with or without growth factors. The concentrations and growth factors added are as described in Example 1. After 1, 5, or 7 days of storage, the metanephroi were transplanted as described in Example 1. Development of the metanephori into functional chimeric kidneys was determined as outlined in Example 1.

### Results

As shown in Table 4, none of the metanephroi preserved for 5 or 7 days in UW solution minus growth factors engrafted. In contrast, 1 out 5 metanephroi preserved for 5 or 7 days in UW solution plus growth factors developed into a functional kidney.

TABLE 4
Weights, urine volumes and inulin clearances of metanephroi (Met)

| | | | Preserved for | | Preserved for | |
|---|---|---|---|---|---|---|
| Growth Factors | Preserved for 1 Day | | 5 Days | | 7 Days | |
| (GE) | None | GF | None | GF | None | GF |
| Number | 5 | 3 | 0 | 1 | 0 | 1 |
| Met Weight | 70 ± 12 | 67 ± 4.6 | NA** | 63 | NA | 23 |
| (mg) | | | | | | |
| Host Weight | 272 ± 7.8 | 286 ± 12 | NA | 250 | NA | 317 |
| (g) | | | | | | |
| Urine vol. | 112 ± 16 | 234 ± 60* | NA | 40 | NA | 82 |
| (μl/hr) | | | | | | |
| Inulin | 0.68 ± 0.1 | 1.1 ± 0.4* | NA | 0.3 | NA | 0.5 |
| Clearance | | | | | | |
| (μl/mim/ | | | | | | |
| 100 mg) | | | | | | |

*GF > None; p < 0.05, Student's t test.
**NA = not applicable

We claim:

1. A method of preserving embryonic metanephric tissue comprising contacting said tissue with an EMT preservation solution.

2. A method according to claim 1 wherein the temperature of said preservation solution is between 0 to 4° C.

3. A method according to claim 1 wherein said preservation solution further comprises one or more growth factors.

4. A method according to claim 3 wherein said growth factors are selected from the group consisting of recombinant human insulin-like growth factor I, recombinant human insulin-like growth factor II, recombinant human transforming growth factor α, recombinant human hepatocyte growth factor, recombinant human vascular endothelial growth factor, recombinant human basic fibroblast growth factor, recombinant human nerve growth factor, retinoic acid, corticotropin releasing hormone, Tamm Horsfall protein, prostaglandin E1, and iron-saturated transferrin.

5. A method according to claim 1 wherein said metanephroi is transplanted after contacting an EMT preservation solution.

6. A method according to claim 5 wherein said EMT preservation solution further comprises one or more growth factors.

7. A method according to claim 5 or 6 wherein said contact ranges from less than an hour to 7 days.

8. A composition comprising an EMT preservation solution in combination with one or more growth factors.

9. A composition according to claim 8 wherein said growth factors are selected from the group consisting of recombinant human insulin-like growth factor I, recombinant human insulin-like growth factor II, recombinant human transforming growth factor α, recombinant human hepatocyte growth factor, recombinant human vascular endothelial growth factor, recombinant human basic fibroblast growth factor, recombinant human nerve growth factor, retinoic acid, corticotropin releasing hormone, Tamm Horsfall protein, prostaglandin E1, and iron-saturated transferrin.

10. A composition according to claim 9 wherein said EMT preservation solution is University of Wisconsin preservation solution comprising at least two of said growth factors.

11. A composition comprising embryonic metanephroi and an EMT preservation solution.

12. A composition according to claim 11 wherein said EMT preservation solution further comprises one or more growth factors.
